(19) United States
(12) Patent Application Publication
(10) Pub. No.: US 2009/0031458 A1
(43) Pub. Date: Jan. 29, 2009

(54) Thaumatin From Transgenic Barley

(75) Inventors: Rainer Stahl, Bonn (DE); Renate Luhrs, Bonn (DE); Harald Dargatz, Andernach (DE)

Correspondence Address: CONNOLLY BOVE LODGE & HUTZ, LLP, P O BOX 2207, WILMINGTON, DE, 19899, US

(73) Assignee: Maltagen Forschung GmbH, Andernach (DE)

(21) Appl. No.: 11/989,675

(22) PCT Filed: Jul. 28, 2006
(86) PCT No.: PCT/EP2006/007525

§ 371 Date: Aug. 14, 2008

(30) Foreign Application Priority Data
Jul. 30, 2005 (DE) 10 2005 035 888.8

(51) Int. Cl. A01H 5/00; C12N 15/82; C12N 5/10; A01H 5/10; C12N 15/29

(52) U.S. Cl. 800/320; 435/320.1; 435/419; 536/23.6

(57) ABSTRACT

The invention relates to transgenic plants from barley containing more than 2 grams of thaumatin in 1 kg of kernel material, to extracts and products obtained from it, especially malt and flour products, including food for humans and animal feed.

The present invention relates to thaumatin from transgenic plants from the cultivated plant barley (Hordeum vulgare), to extracts and products obtained from it, especially malt and flour products, including food for humans and animal feed.

Obesity and diabetes have led consumers in industrialized countries to increasingly seek out products containing “low-caloric” sweeteners. In this context, precisely the “intense sweeteners” are playing a growing role (Gibbs B. F. et al. (1996) Nutrition Research 16 (9): 1619-1630; Faus I. (2000) Appl. Microbiol. Biotechnol. 53: 145-151). Unlike the sugar alcohols, the sweeteners are either not metabolized (for example, cyclamate, saccharin) or, due to their sweetening power, which is 200 to 3000 times that of sucrose, they are used in such small quantities that the calorific value is negligible (for instance, aspartame, thaumatin). At the present time, primarily synthetic sweeteners are employed worldwide in the production of food for humans and animal feed. However, in order to prevent health-related side effects, restrictions on the quantity (ADI=acceptable daily intake) apply for these sweeteners in many countries. In contrast, the natural sweetener thaumatin, a mixture of proteins from the katemfe fruit (Thaumatococcus daniellii), is considered safe. In accordance with the various guidelines, thaumatin (E959) is approved in the European Union (94/35/EC; 95/2/EC, 2003/115/EG, 88/388/EC), Switzerland, U.S.A., Canada, Israel, Mexico, Japan, Korea, Singapore, Hong Kong, Australia, New Zealand and South Africa.

Thaumatin is also classified as GRAS (generally recognized as safe, an FDA criterion). Thaumatin has also been tested by JEFCA (Joint Expert Committee on Food Additives of the FAO/WHO) as well as by the European Safety Authority (EFSA) (Gibbs (supra); Faus I. (supra)). Six isoforms of the protein have been found in the fruit of Thaumatococcus daniellii, all of which are sweet (thaumatin 1, II, III, a, b, c,). Thaumatin I and II are the main forms, which only differ from each other in 4 amino acids and which each consist of 207 amino acids. The three-dimensional structure of thaumatin I consists of three domains, whereby domains II and III are characterized by regions rich in disulfide bridges. Altogether, a correctly folded thaumatin forms 8 disulfide bridges (from De Vos et al., Proc. Natl. Acad. Sci. USA (1985) 82: 1406-1409; Ogata et al., J. Mol. Biol. (1992) 228: 893-908; Masuda et al., Biotechnol. Bioeng. (2004) 85: 761-769). The various known preprothaumatins are characterized by an N-terminal and a C-terminal elongation that probably function as signals for the compartmentalization in the plant cell. More in-depth studies, however, are not available (Van der Wel and Loewe, Eur. J. Biochem, (1972) 272: 14810-14816; Witty M., Biotechnol. Lett. (1990) 12: 131-136; Gibbs B. F. (supra); Faus I. (supra); Faus I. and Sisniega H. (2002) “Sweet tasting proteins” in Biopolymers, Steinbüchel A., Fahestock S. (eds.); Kant R., Nutrition Journal (2005) 4 (5): 1-6). The sweetening power of thaumatin is 2000 to 3000 times higher than that of sucrose (on the basis of the weight, and approximately 100,000 times higher on a molar basis), although the sweetness of thaumatin is perceived with a certain delay. At very high concentrations, thaumatin, like other plant-based sweeteners, can have a licorice-like aftertaste (Faus I. (2000) Appl. Microbiol. Biotechnol. 53: 145-151). In order to improve the taste, several mutants have been produced (Weickmann J. L., Lee J. H., Blair L. C., Ghosh-Dastidar P., Koduri R. K. (1989) in Gremby T. H., ed., “Progress in sweeteners”, New York: Elsevier, pages 47-69; EP 0396741, U.S. Pat. No. 5,221,624, WO 90/05775).

The heat stability of thaumatin depends on the matrix. Higginbotham has reported that the sweetness of thaumatin I is retained after being boiled for one hour, followed by cooling off subsequently (even at a pH value of less than 5.5) (Higginbotham J. D. (1986) in Nabors L. O., Gelardi R. C., eds “Alternative Sweeteners”, Marcel Dekker, New York: 103-134). In any case, thaumatin is stable under pasteurizing conditions (Gibbs B. F. et al., Nutrition Research (1996), 16 (9): 1619-1630.) In purified form, the protein aggregates and loses its sweetening power at a temperature of 70° C. [158° F.] and at a pH of 7.0 (Kaneko R. and Kitabatake N., J. Agric. Food Chem. (1999) 47 (12): 4950-4955). In freeze-dried form, thaumatin is stable, soluble in water and alcohol and thus suitable for the production of beverages.

In addition to its sweetening power, thaumatin also has flavor-enhancing properties (Gibbs et al., Nutrition Research (1996) 16 (9): 1619-1630; U.S. Pat. No. 6,420,527). Consequently, it is highly suitable to mask the foreign taste of some sweeteners and to improve the mouth feel. Thaumatin is added to round off the flavor of sugar alcohols and sweetener mixtures (EP 0681789, EP 0847242, U.S. Pat. No. 6,562,392, U.S. Pat. No. 6,555,146, WO 09708958, EP 0658340, WO 0106872, EP 1198181). In addition, irrespective of the sweetness, thaumatin is also employed purely for purposes of enhancing the flavor of gravies and meats (Gibbs et al. (supra), U.S. Pat. No. 6,420,527).

Thaumatococcus daniellii, a shrub from whose fruit thaumatin is extracted, grows in the rainforests of West Africa. The cultivation of this shrub is very difficult since the plants have to be shaded and have to be pollinated exclusively by specific insects. Therefore, only one-fourth of the plants in open fields bear fruit (Faus (supra)).

Consequently, attempts are repeatedly being made to cultivate the tropical plant Thaumatococcus daniellii more efficiently. However, the production of thaumatin makes use primarily of fruit gathered in forests. The highest percentage of thaumatin, 20% to 40% of the dry weight, is concentrated in the aril of the fruit. The creamy-white aril is located at the apex of the seed and it is surrounded by a transparent gel. The mechanical separation of this tissue from the seed is relatively arduous.

Thaumatin can be extracted from the fruit by means of simple aqueous extraction (Van der Wel and Loewe, Eur. J. Biochem. (1972) 31: 221-225). Ultrafiltration or ion-exchange chromatography can be employed to further purify thaumatin (U.S. Pat. No. 4,221,704). The purification is made more difficult by the gel that surrounds the aril, since it swells up a great deal during aqueous extraction. Various methods are known in order to simplify the purification of thaumatin from Thaumatococcus daniellii. Freeze-drying the fruit makes it easier, for example, to remove the aril, and salts or acids can be employed to prevent the gel from swelling up so that thaumatin can be extracted more efficiently (U.S. Pat. No. 4,011,206, U.S. Pat. No. 4,221,704, EP 00003911). Consequently, the conventional extraction and concentration, along with the isolation of thaumatin, entail a great deal of effort.

Owing to the limited availability of thaumatin, this sweetener has only been utilized in very special products so far. Thaumatin, however, is not only suitable for rounding off the flavor of sweetener mixtures (e.g. in NATREEN: WO 0106872, EP 1197181), but it also lends itself very well for sweetening beverages (soft drinks, alcoholic beverages, coffee and tea beverages), milk products (yoghurt, desserts), candy, chocolate, chewing gum, chewable tablets, etc. and also as a flavor enhancer in gravies and diet meals. Thaumatin is also added as a flavoring agent to animal feed (primarily for pet food) (Etheridge K. (1994) in Witty: M., Higginbotham J. D., eds. Thaumatin, Boca Raton: CRC Press, pp. 47-59, Tate & Lyle: GB 2185674, U.S. Pat. No. 6,251,464, EP 0929233, WO 9803082, U.S. Pat. No. 5,932,438, EP 0684312).

Due to the extreme sweetening power of the intense sweeteners, they are only employed in extremely small doses. As a result, they entail the drawback that they cannot replace the volume of sugar or sugar substitutes. Accordingly, diet products often male use of sugar alcohols as fillers, which are additionally sweetened by the sweeteners. Sugar alcohols, however, can impair the consistency and taste of certain products (for instance, baked goods). Moreover, sugar alcohols contain calories, which limits their suitability for the manufacture of low-calorie products.

Consequently, there is a great need to produce thaumatin by means of biotechnology. There is also a great need to provide thaumatin as an endogenous product for food for humans and animal feed.

The state of the art so far includes biotechnological methods to extract and concentrate thaumatin in various organisms. Due to the limited availability of the raw material, a number of attempts have been made to produce thaumatin in microorganisms and higher organisms by means of biotechnology. In most cases, not only is the yield uneconomically low but moreover, the thaumatin is not sweet because of its changed folding. It is a known procedure to produce active thaumatin in E. coli, Bacillus subtilis, Streptomyces levidans, Aspergillus awamori, Saccharomyces cerevisiae, Klyveromyces lactis, Pichia pastoris, etc. by adapting the codon usage or by producing various mutants (Weickmann J. L., Lee J. H., Blair L. C., Ghosh-Dastidar P., Koduri R. K. (1989) in Gremby T. H., ed., “Progress in sweeteners”, New York: Elsevier, pp. 47-69, U.S. Pat. No. 4,891,316, EP 0003911, EP0054330, EP0096910, WO8404538, U.S. Pat. No. 5,221,624, EP0396741, EP 0255823, WO 9005775, WO 8703007, U.S. Pat. No. 5,932,438, EP 0684312).

TABLE 1
Overview of biotechnological methods to produce thaumatin in several organisms.

| | | | |
|---|---|---|---|
| | | Sweet | |
| Organism | Yield | phenotype | Reference |

Microorganisms:

| | | | |
|---|---|---|---|
| E. coli | very low | no | Edens et al. (1982), Gene 18 (1): 1-12, |
| | | | Faus et al. (1996), Biochem. Biophys. |
| | | | Res. Commun. 229 (1): 121-127 |
| Bacillus subtilis | 1 mg/l | yes | Illingworth et al. (1988), Biotechnol. |
| | | | Lett. 10: 587-592 |
| Streptomyces lividans | 0.2 mg/l | ? | Illingworth et al. (1989), J. Ind. Micro- |
| | | | biol. 4: 37-42 |
| S. cerevisiae | low | no | Lee et al. (1988), Biochemistry 27: |
| | | | 5101-5107 |
| | 0.3 g/l | yes | Weickman et al. (1994) in Witty M. |
| | | | Higgingbotham, eds. “Thaumatin”, CRC |
| | | | Press, Boca Raton, FL: 151-169 |
| Klyveromyces lactis | low | no | Edens and van der Wel (1985), Trends |
| | | | Biotechnol. 3 (3): 61-63 |
| Aspergillus awamori | 70 mg/l | yes | Moralejo et al. (2000), Appl. Microbiol. |
| | | | Biotechnol. 54: 772-777, Lombrana et |
| | | | al. (2004), Appl. Envir. Microbiol. 70 |
| | | | (9): 5145 |
| Pichia pastoris | 25 mg/l | yes | Masuda et al. (2004), Biotechnol. |
| | | | Bioeng. 85 (7): 761-769 |

Plants:

| | | | |
|---|---|---|---|
| potato | 0.2-2 mg/kg | yes | Witty (1989), Nucleic Acids Res. 17 (8): |
| | | | 3312, Witty, Methods Enzymol. (1992) |
| | | | 216: 441-447 |
| pear | very low | ? | Lebedev et al. (2002), Acta Hort. 596: |
| | | | 199-207 |
| tomato | very low | yes | Bartoszesi et al. (2003), Plant Breeding |
| | | | 122: 347-351 |
| cucumber | very low | yes | Gajc-Wolska et al. (2003), Acta Hort. |
| | | | 604: 449-451 |
| apple | very low | yes (leaves) | Dolgov et al. (2004), Acta Hort. 663: |
| | | | 507-510 |
| tobacco | 1.5 g/kg of | ? | Icon Genetics: transientes, virales |
| | leaf material | | Expressionssystem [transient, viral |
| | | | expression system] |
| | | | (www.icongenetics.com) |

The idea to produce the natural sweeteners monellin and thaumatin directly in the edible tissue of higher plants was already published by Fischer et al. (WO 9201790, EP 0540651). Monellin is likewise a protein from a tropical plant (Dioscoreophyllum comminsii) that imparts an intense sweet taste. The examples showed that, in the case of tomatoes, trans-formation with the monellin gene under control of the fruit-specific E8 promoter can yield a sweeter flavor in the fruit. Moreover, a sweeter taste was found in all of the tissues of lettuce that had been transformed with the monellin gene under control of the constitutive CaMV35S promoter. This document, however, does not contain any reproducible disclosure pertaining to transgenic monocotyledonous plants, especially to a cultivated plant such as a cereal grain like barley, containing thaumatin. Furthermore, the disclosed promoters are not suitable to obtain thaumatin in transgenic cereal grains. Moreover, back 1992, there were not yet any reproducible transformation protocols for monocotyledonous plant species (e.g. for rice: Hiei et al., Plant J. (1994) 6: 271-282; for an overview: Christou P., “Rice transformation: bombardment”, Plant Mol. Biol. (1997) 35: 197-203; Hiei et al., “Trans-formation of rice mediated by Agrobacterium tumefaciens”, Plant Mol. Biol, (1997) 35: 205-218); for corn, for instance, only as of 1994 via biolistic DNA transfer (Klein, Biotechnology Agricult. Forestry (1994) 25: 241-251; Brettschneider et al., Theor. Appl. Genet. (1997) 94: 737-748) via Agrobacterium tumefaciens only as of 1996: Ishida et al., Nature Biotechnology (1996) 14 (6): 745-750); the method for the transformation of rye (De la Pena et al., Nature (1988) 325: 274-276) was withdrawn for not being reproducible (Langridge et al., Plant J. (1992) 631-638); the reproducible production of transgenic rye plants via a biolistic approach only succeeded in 1994 (Castillo et al., Biotechnology (1994) 13: 1366-1371); via Agrobacterium tumefaciens only as of 2003 (Popelka et al., Molecular Breeding (2003) 11: 203-211); for wheat, appropriate transformation protocols were published in 1993 and 1994 as well as 1997 (biolistic DNA transfer: Vasil et al., Biotechnology (1993) 11: 1553-1558; Weeks et al., Plant Physiol. (1993) 102: 1077-1084; Becker et al., Plant J. (1994) 5: 299-307), Transformation über Agrobacterium tumefaciens [Transformation via Agrobacterium tumefaciens] Cheng et al., Plant Physiol. (1997) 115: 971-980); the production of transgenic oat plants was achieved for the first time in 1992 (Somers et atl., Biotechnology (December 1992) 10: 1589-1594); the first transgenic millet was obtained in 1993 and 2002 (Sorghum: Casa et al., Proc. Natl. Acad. Sci. USA (1993) 90 (23): 11212-11216; Pennisetum: Girgi et al., Mol. Breed. (2002) 10 (4): 243-252); barley could only be reproducibly transformed in 1994 via biolistic DNA transfer (Wan and Lemaux, Plant Physiol. (1994) 104: 37-48; Rital et al., Plant Mol. Biol. (1994) 24: 317-325), via Agrobacterium tumefaciens in 1997 (Tingay et al., Plant J. (1997) 11: 1369-1376.)).

Witty (1989, supra) transformed the thaumatin II gene in potatoes. Potatoes, however, are suitable neither for an improvement of the flavor nor for the cost-effective production of thaumatin. Due to the high sweetening power of thaumatin, the sweetness can already be tasted at a concentration of 10⁻⁸ M, which corresponds to about 2 μg/l. The yield in potatoes is between 2 μg/kg and 2 mg/kg (Witty, Methods Enzymol. (1992) 216: 441-447).

In more recent studies, tomatoes, cucumbers, apples and pears were transformed with the thaumatin gene in order to improve the flavor (Table 1). In the case of tomatoes and cucumber, a sweeter taste was confirmed in the fruits. In other words, these organisms are not suitable as bioreactors for thaumatin, but rather, are only employed to improve the flavor (Bartoszewski et al., Plant Breeding (2003) 122, 347-351; Gajc-Wolska et al., Acta Horticulturac (2003) 604: 449-451; Dolgov et al. (2004), Acta Hort (ISHS) 596: 199-202; Lebedev et al. (2002) Acta Hort. (ISHS) 596: 199-202).

Recently, a viral expression system was employed in tobacco in order to produce thaumatin (www.icogenetics.com). Up to 1.5 g/kg of leaf material were produced. This system, however, is unsuitable for producing thaumatin on a large scale since the plants are not trans-formed in a stable manner, but rather, the viral vectors are vacuum-filtrated into the leaves via agro-bacteria (transient expression). Moreover, tobacco contains numerous toxic ingredients so that the extraction of the sweetener for the production of food for humans and animal feed is not an option.

For this reason, it is the objective of the present invention to produce thaumatin by means of a biotechnical method that results in cost-effective yields and in suitable stability and sweetness.

In order to achieve this objective, transgenic plants from the cultivated plant barley (Hordeum vulgare) (also referred to below as “transgenic plant from barley”) has been selected as the bioreactor.

Up until now, thaumatin has not been successfully expressed in such inventive transgenic plants from barley. Moreover, it was not possible to produce thaumatin with sufficient sweetness and stability in such inventive transgenic plants from barley Regarding the non-reproducible disclosure content of WO 9201790 concerning this aspect, reference is made to the elaborations above.

All that is known is the transformation of the gene for the sweet protein brazzein from the plant Pentadiplandra brazzeana in monocotyledonous corn. The yields of brazzein in corn amount to approximately 4% of the soluble protein, corresponding to about 1 g/kg of the kernel material (Faus (2000) Appl. Microbiol. Biotechnol. 53: 145-15; Lamphear et al. (2005) Plant Biotechnol. Journal 3 (1) 103-114).

Surprisingly, thaumatin in the inventive transgenic plants from barley can be expressed in high yields while the sweetening power and sufficient stability are retained (so-called “active thaumatin”). The yields obtained are of more than 2 g/kg, 3 g/kg or more (kernel material) of active thaumatin. This corresponds to the sweetening power of about 3 kg to 9 kg of sucrose.

The sweetness and stability of endogenously expressed thaumatin can be obtained according to the invention by means of the correct folding of the thaumatin—that is to say, of the active thaumatin—in the inventive transgenic plant from barley.

The cited high yields are achieved in the endosperm of the inventive transgenic plant from barley.

Consequently, the invention relates to an inventive transgenic plant from barley (Hordeum vulgare) containing more than 2 grams of thaumatin or active thaumatin or a protein having an activity for thaumatin in 1 kg of kernel material. Therefore, for one thing, the invention also relates to suitable polynucleotides or nucleic acids or other analogs (e.g. T-DNA) that code for thaumatin or for a protein having an activity for thaumatin and that are suitable according to the invention to transform barley by means of a vector, thereby obtaining such transgenic plants from barley. Examples of suitable vectors are described in Hellen and Mullineaux (2000) “A guide to Agrobacterium binary TI vectors”. Trends in Plant Science 5(19): 446-451. Examples of vectors are also disclosed in the examples and figures.

Consequently, the invention likewise relates to an inventive transgenic plant from barley (Hordeum vulgare) having at least one polynucleotide that codes for thaumatin and that is stably integrated into the genome after its transformation, particularly by means of promoters and optionally other secondary sequences (enhancers, terminators, signal sequences, etc.) that serve for the controlled expression of thaumatin.

In another preferred embodiment of the invention, thaumatin is systematically directed (targeting) into the endoplasmic reticulum (ER) of the endosperm of the kernel material or seed, especially for purposes of attaining the correct folding of the thaumatin.

It is known that the protein disulfide isomerases play a major role in the correct expression of the three-dimensional structure and thus in the stability of the proteins. The protein disulfide isomerases catalyze the rearrangement of the disulfide bridges during the folding of the proteins (Wilkinson and Gilbert, Biochim. Biophys. Acta (2004) 1699: 35-44; Sitia and Molteni Sci. STKE (2004) 239: 27). The significance of these enzymes for the stability and thus concentration of thaumatin is shown by experiments with Aspergillus awamori. Five times higher yields of thaumatin were obtained in this non-plant organism, for instance, by means of a defined over-expression of the protein disulfide isomerase (PDI) (Moralejo et al., Mol. Genet. Genomics (2001) 266: 246-253).

Disulfide bridges and thus the correct, biologically active protein conformation fundamentally cannot form under the conditions that are maintained in the interior of the cells. Therefore, the folding of the proteins and the structure of the disulfide bridge patterns take place in the eukaryotic cells under partially oxidizing conditions in certain compartments, for example, in the endoplasmic reticulum (ER). In this context, the protein disulfide isomerases (PDI) play a major role in the formation of the complex three-dimensional structures of the proteins (Boston et al., Plant Mol. Biol. (1996) 32: 191-222; Wilkinson and Gilbert, Biochim. Biophys. Acta (2004) 1699: 35-44).

During the kernel development in cereal grains, the protein disulfide isomerases play a role in the correct folding and accumulation of the storage proteins (Shimoni et al., Plant Physiol. (1995) 108: 327-335; Li and Larkins, Plant Mol. Biol. (1996) 30: 673-882; Takemoto et al., Plant Physiol. (2002) 128: 1212-1222). A concentration of protein disulfide isomerases was observed in a proteome analysis of barley seeds at various stages of the kernel development (Finnie et al., Plant Physiol. (2002) 129: 1308-1319).

The systematic targeting of heterologous proteins by means of signal sequences of the storage proteins, for instance, those of the hordein D gene from barley, is a known procedure (Horvath et al., Proc. Natl. Acad. Sci. USA (2000) 97: 1914-1919). What is not known, however, is the correct endoplasmic reticulum (ER) targeting of the thaumatin, optionally by means of the thaumatin-inherent signal sequences together with non-thaumatin signal sequences as well as the correct processing of these thaumatin-inherent N-terminal and C-terminal sequences in the inventive transgenic plants from barley (Hordeum vulgare). The result obtained is a correctly folded thaumatin (active thaumatin), which is essential for the invention.

Therefore, in another embodiment of the invention, it is preferred for inventive polynucleotides that code for thaumatin to be transformed under control of an endosperm-specific promoter. Such endosperm-specific promoters are non-terminal promoters of the storage proteins from barley, for instance, hordein-B, -C, -D, -G promoters (Hordein-D, see SEQ ID NO: 1, Forde et al., Nucleic Acids Res. (1985) 25: 7327-7339; Sörensen et al., Mol. Gen. Genet. (1996) 250: 750-760; Horvath et al. Proc. Natl. Acad. Sci. USA (2000) 97: 1914-1919; Cameron-Mill and Brandt, Plant Mol. Biol. (1988) 11: 449-461); or gamma hordothionine promoters such as those disclosed, for example, in PCT/EP2004/009762; or from rice, for instance the Gt1 promoter (Okita et al., J. Biol. Chem. (1988) 264: 12573-12581; Leisy et al., Plant Mol. Biol. (1990) 14: 41-50); or from wheat, for instance, the Glu-lD-1 promoter (Zhang et al., Theor. Appl. Genet. (2003) 106: 1139-1146).

For this reason, preference is given to an endosperm-specific promoter whereby, according to the invention, protein disulfide isomerases are likewise present to allow the correct folding of thaumatin. Precisely this combination allows the desired high yields in the endosperm of the transgenic plants according to the invention, particularly preferably barley.

In another embodiment, the invention also relates to the use of signal sequences for the systematic targeting of thaumatin into the endoplasmic reticulum (ER) of the endosperm cells as well as the correct accumulation in “protein bodies” of these endosperm cells. Signal sequences of the storage proteins of barley, for example, hordein B, hordein C, hordein D, hordein G can preferably be used for this purpose (Forde et al., Nucleic Acids Res. (1985) 25: 7327-7339; Sörensen et al., Mol. Gen. Genet. (1996) 250: 750-760; Horvath et al., Proc. Natl. Acad. Sci. USA (2000) 97: 1914-1919; Cameron-Mill and Brandt, Plant Mol. Biol. (1988) 11: 449-461) or the signal sequence of the gamma hordothionine from barley (PCT/EP2004/009762). Such signal sequences are, for instance, non-terminal SEQ ID NO: 2-8 as well as its expression products SEQ ID NO: 9-15.

Surprisingly, the thaumatin-inherent N-terminal and C-terminal signal sequences likewise lead to systematic targeting into the ER and to the correct accumulation in the protein bodies.

Likewise surprising is the fact that the signal sequences are correctly processed by barley-specific proteases. Such signal sequences are, for instance, non-terminal SEQ ID NO: 16-17 as well as its expression products SEQ ID NO: 18-19.

Consequently, the invention relates to an inventive polynucleotide that codes for thaumatin or for a protein having an activity for thaumatin, which is the object of a construct or of an expression cassette containing promoters and, if applicable, other secondary sequences for the controlled expression of thaumatin, whereby the promoter is preferably an endosperm-specific promoter (supra) as well as the cited signal sequences (supra) for targeting into the endoplasmic reticulum (ER) of the endosperm cells.

The invention likewise relates to a vector containing such an inventive construct or expression cassette as well as to an inventive transgenic plant from barley produced by means of such a vector. Examples of such a construct or expression cassette are SEQ ID NO: 20-21.

According to the invention, transgenic plants from barley (Hordeum vulgare), plant cells or protoplasts are obtained that have at least one inventive polynucleotide stably integrated into the genome after its transformation. Consequently, the invention likewise relates to seeds for planting purposes obtained from the transformed plants or transgenic plants.

The invention also relates to kernel material or seeds of a transgenic plant from barley (Hordeum vulgare) containing more than 2 grams or more than 3 grams of thaumatin in 1 kg of kernel material.

As defined by this invention, thaumatin is a protein sequence containing 207 amino acids and having 8 disulfide bridges that occur natively in the isoforms 1, II. Known sequences are, in particular, SEQ ID NO: 22-24 as well as the coding sequences SEQ ID NO: 25-27. Likewise included are proteins having an activity for thaumatin.

According to the invention, it is now possible for the first time to produce the native amino acid sequence of thaumatin I, also with the correct folding, in a heterologous organism, or in a plant. Up until now, preferably thaumatin II has been heterologously expressed in the various non-monocotyledonous organisms (see Table 1). So far, thaumatin I has only been recombinantly produced in the form of mutated variants (U.S. Pat. No. 5,221,624, WO 9005775, EP 0540651, Lee et al., Biochemistry (1988) 27: 5101-5107).

It is known that thaumatin I has a higher sweetening power (Masuda et al., Biotechnol. Bioeng. (2004) 85: 761-769) as well as a higher thermal stability (Higginbotham J. D. (1986) in: Nabors L. O., Gelardi R. C., eds. “Alternative Sweeteners”, Marcel Dekker New York: 103-134).

In a special embodiment of the invention, the invention relates to a new amino acid sequence according to SEQ ID NO: 28, whereby thaumatin I can be isolated with the specific N-terminus and C-terminus from a transgenic plant according to the invention, especially barley. The presented sequence is an active thaumatin.

Therefore, the invention also relates to polynucleotides that code for the amino acid according to SEQ ID NO: 28, particularly according to DNA SEQ ID NO: 29.

The term “transgenic plant” relates to plants that were produced by means of recombinant genetic engineering and/or microbiological methods and not by means of conventional breeding, and that contain at least one polynucleotide according to the invention that codes for thaumatin. Methods for the production of transgenic plants have been described (Tingay S., McElroy D., Kalla R., Fieg S., Wang M., Thorton S. and Brettel R. (1997): “Agrobacterium tumefaciens-mediated barley transformation”. Plant Journal 11, 1369-1376; Wan Y. and Lemaux P. (1994): “Generation of a large number of independently transformed fertile barley plants”. Plant Physiol. 104; 37-48, Stahl R., H. Horvath, J. Van Fleet, M. Voetz, D. von Wettstein & N. Wolf (2002) “T-DNA integration into the barley genome from single and double cassette vectors”. Proc. Natl Acad. Sci. USA 99, 2146-2151; Horvath H., J. Huang, O. T. Wong & D. von Wettstein (2002) “Experiences with genetic transformation of barley and characteristics of transgenic plants” in: Barley Science, G. A. Slafer, J. L. Molina-Cano, R. Savin, J. L. Araus & I. Romagosa eds. The Harworth Press, New York 2002 pp. 143-176; Horvath H., L. G. Jensen, O. T. Wong, E. Kohl, S. E. Ullrich, J. Cochran, C. G. Kannangara & D. von Wettstein (2001) “Stability of transgene expression, field performance and recombination breeding of transformed barley lines”. Theor. Appl. Genet. 102, 1-11; Wettstein D. von, G. Mikhaylenko, J. A. Froseth & C. G. Kannangara (2000) “Improved barley broiler feed with transgenic malt containing heat-stable (1,3-1,4)-beta-glucanase”. Proc. Natl. Acad. Sci. USA 97, 13512-13517; Horvath H., J. Huang, O. T. Wong, E. Kohl, T. Okita, C. G. Kannangara & D. von Wettstein (2000) “The production of recombinant proteins in transgenic barley grains”. Proc. Natl. Acad. Sci. USA 97, 1914-1919; Mayerhofer, R., Koncz-Kalman, Z., Nawrath, C., Bakkeren, G., Crameri, A., Angelis, K., Redei, G P., Schell, J., Hohn, B. & Koncz, C. (1991) EMBO J. 10, 697-704 “T-DNA integration: a mode of illegitimate recombination in plants”; Deblaere R., Bytebier B., De Greve H., Deboeck F., Schell M., Van Montagu M., Leemans J.; “Efficient octopine Ti plasmid-derived vectors for Agrobacterium-mediated gene transfer to plants”; Nucleic Acids Res. 13: 4777-4788 (1985)).

The inventive transgenic plants from barley (Hordeum vulgare) can be easily malted to form malt. Roasting methods can be employed to impart the malt with additional flavor components. Malt is already an integral part of many food products for humans and of animal feed. Malt can constitute the filler for the use of thaumatin and consequently, it is extraordinarily well-suited as food for humans or animal feed containing endogenous thaumatin.

Therefore, the invention also relates to a flour product or malt product, also to extracts and powders, or to food for humans and animal feed containing kernel material from the inventive transgenic plant from barley.

Another subject matter of the invention is food products, low-calorie or diet foodstuffs comprising beverages, beer, bread, coffee or cocoa beverages, sweets, ice cream, readymade meals, milk and yoghurt products, cereal products, candy, chocolate, chewing gum and chewable tablets, baked goods, cakes, gravies, dressing containing an extract or a flour product or malt product and its extracts, all made of an inventive transgenic plant from barley (Hordeum vulgare) or from its kernel material.

Production of a malt powder from a transgenic plant from barley (Hordeum vulgare): the malt powder-made of the transgenic barley according to the invention combines the properties of thaumatin (sweetening and flavor enhancement) with the pleasant flavor of malt. The powder is highly suitable as an additive to food products such as, for instance, coffee or cocoa beverages, sweets, ice cream, ready-made meals, milk and yoghurt products, cereal products, gravies, dressings as well as for animal feed.

Since thaumatin in malt exhibits very high heat resistance, it is likewise very well-suited for making cookies and cakes. Due to the high sweetening power of the thaumatin barley, there is no need to add any other sweetener to the products. Therefore, malt powder made of thaumatin barley can be used to great advantage to make low-calorie or diet products. The manufacture of these various products is known to the person skilled in the art. For instance, up to 10% malt powder (100 grams of malt per kilogram of baking mix) is added to the baking mix used for cookies. In terms of the sweetening power, the addition of 100 grams of thaumatin malt corresponds to the addition of 300 grams of sucrose per kilogram of baking mix.

Likewise known to the person skilled in the art is the production of beverage powders containing malt (e.g. NESTOMALT, EP 1068807) that are then mixed with hot or cold water or milk. The use of malt from the transgenic barley according to the invention makes it possible to completely dispense with the addition of sugar or sugar substitutes. The thaumatin in the malt not only imparts the sweetness but also a pleasant mouth feel that cannot be imparted by other sweeteners or sugar alcohols.

Flour and malt from the transgenic barley according to the invention can be added directly to animal feed mixtures. One ton of animal feed for piglets is sweetened, for example, with 20 kg to 40 kg of lactose (e.g. in ANILAC, ANIFIT). The sweetening power of lactose is only 0.6 times that of sucrose. A corresponding sweetening can be achieved by adding 1.5 kg to 2.5 kg of the thaumatin malt or flour according to the invention per ton of animal feed for piglets. In particular, flour and malt from the transgenic plants according to the invention can be added to animal feed as a flavor enhancer.

Moreover, the inventive malt from transgenic barley containing thaumatin is suitable for the production of a malt extract, malt syrup or instant malt powder. When it comes to making beverages, the use of a malt extract is suitable to ensure 100% solubility. In this context, the state of the art for the production of malt extracts can be employed to a large extent. The strength of the malt flavor can be varied by an extraction process (for instance, Owades J. L. et al.: “Preparation of a non-alcoholic malt beverage”, U.S. Pat. No. 5,120,557).

Extracts from the malt of the thaumatin barley also provide the ideal basis for making tasty, low-calorie or diet beverages. An aqueous extract combines the low-calorie sweetening power and flavor-enhancing properties of thaumatin with the pleasant taste of the malt extract.

For purposes of simplifying the storage and transportation of the malt extract made of the barley according to the invention, a syrup or extract powder can be made employing known methods under vacuum or else an instant powder can be made by means of freeze-drying.

The obtained malt containing thaumatin lends itself very well for the production of tasty diet beers. Owing to the sweetening power of thaumatin, it is not necessary to add sugar (up to 50 μl) during the production of malt beer. The taste of light beers, which do not retain any sweetness due to the complete fermentation of the maltose, can be considerably improved through the addition of the thaumatin malt. Thus, thaumatin malt allows the production of tasty, low-calorie beers, all in accordance with the German Beer Purity Law for the production of beer.

Malt extract is used not only as the basis for the production of beer but also for the production of various soft drinks (e.g. U.S. Pat. No. 4,001,436, U.S. Pat. No. 5,120,557, U.S. Pat. No. 5,415,885, U.S. Pat. No. 6,534,109).

Since the maltose in the malt extract has only a low sweetening power in comparison to sucrose, glucose or fructose, it is necessary to add sucrose, glucose, fructose or other sweeteners in order to sweeten these beverages (for example, U.S. Pat. No. 5,120,557, U.S. Pat. No. 5,415,885).

In other approaches, the sweetening power of the malt extract is increased by means of the enzymatic conversion of the maltose into glucose. However, other sweeteners have to be added to the beverage (e.g. U.S. Pat. No. 4,001,436, U.S. Pat. No. 6,534,109).

The malt extract containing thaumatin from the transgenic barley according to the invention, in contrast, has the intense sweetening power of thaumatin. Only small amounts of the syrup or instant malt powder have to be added to the beverages in order to attain the desired sweetness. There is no need to add other sweeteners, so that the extract, the syrup, the powder and the instant powder are excellent for the production of low-calorie, diet beverages.

The invention also relates to a flavor enhancer consisting of food or animal feed according to the invention, which is added to any desired food or animal feed.

### EXAMPLES

### Example 1

### Construction of a Hordein-Signal/Thaumatin Construct/Expression Cassette/Vector

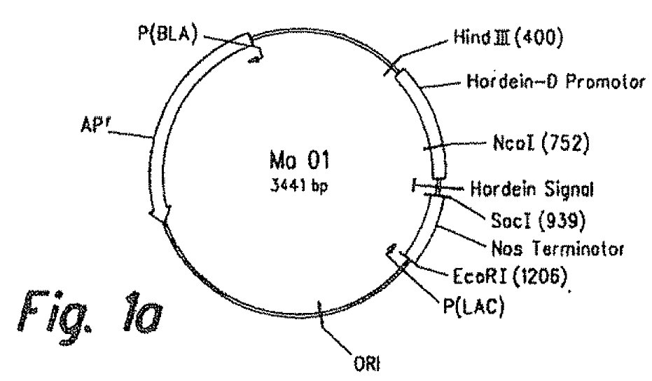

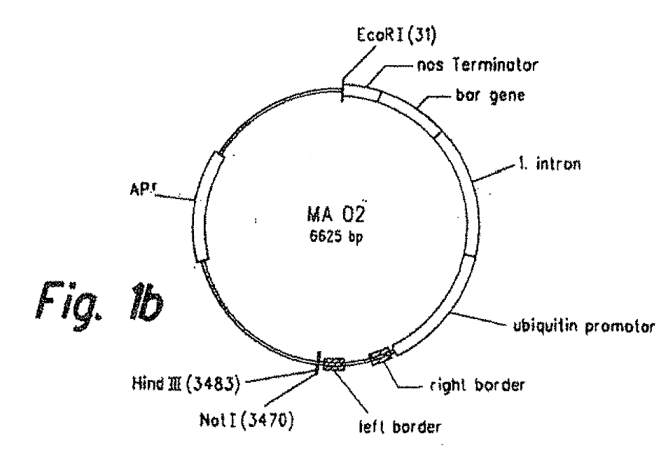

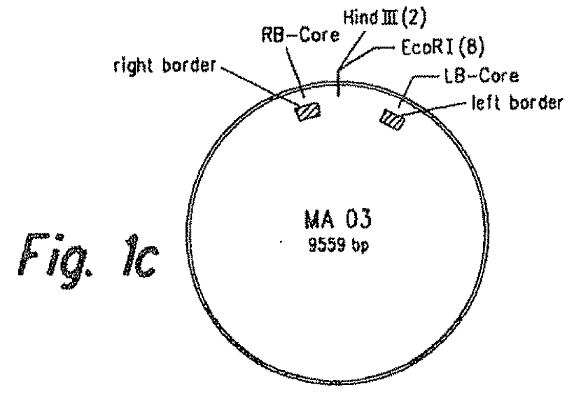

The following work was carried out according to standard methods, as described in Manniatis et al. (supra). The sequence for the mature thaumatin gene was amplified by means of PCR with the extracted DNA (Manniatis et al.) from leaves of Thaumatococcus daniellii. For this purpose, the primers R1004 (SEQ ID NO: 30) at the 5′ end and R1005 (SEQ ID NO: 31) at the 3′ end of the sequence for the mature thaumatin gene were used. By means of splice by overlap, 120 Bp of the sequence at the 3′ end of the sequence of the hordein-D promoter, as well as the sequence for the hordein-D signal peptide were attached to the GC-optimized thaumatin sequence. This PCR fragment was cloned into the SmaI restriction site of pUC18. A fragment with NcoI/SstI was, in turn, cut out of this and cloned into an NcoI/SstI cut vector MA 01 (FIG. 1a), which contains the hordein-D promoter, followed by an SstI restriction site and by the Nos terminator. This gave rise to a construct containing a fragment consisting of hordein-D promoter, hordein-D signal peptide, thaumatin gene and Nos terminator. A fragment with EcoRI and HindIII was, in turn, cut out of this construct. The EcoRi restriction site was filled by means of T4 polymerase and deoxy nucleotides before the cleavage with HindIII. This fragment was subsequently cloned into the vector MA 02 (FIG. 1b) which had been cut with NotI, followed by a filling reaction with T4 polymerase and deoxy nucleotides and, in another step, cut with HindIII Subsequently, a fragment was cut out of this with EcoRI and HindIII and cloned into MA 03 (FIG. 1c). After the transformation of E. coli with the resulting vector and with a plasmid preparation, this plasmid (RS 607, FIG. 1d) was transformed into AGL 1 cells which, in turn, were utilized for the transformation of immature barley embryos. The barley trans-formation was carried out according to Tingay et al. (supra).

### Example 2

### Construction of a Thaumatin-Signal/Thaumatin/C-Terminal Thaumatin-Signal Construct/Expression Cassette/Vector

The following work was carried out according to standard methods, as described in Manniatis et al. (supra). The sequence for the thaumatin gene, including the sequences for the N-terminal and C-terminal signal peptides, was amplified by means of PCR with the extracted DNA (Manniatis et al., supra) from leaves of Thaumatococcus daniellii. The primers R1013 (SEQ ID NO: 32) at the 5′ end of the sequence for the N-terminal signal peptide and R1014 (SEQ ID NO: 33) at the 3′ end of the sequence for the C-terminal signal peptide of the thaumatin were used for this purpose. Another PCR, employing splice by overlap, served to attach 120 Bp of the 3′ sequence of the hordein-D promoter to the sequence for the mature thaumatin plus the sequences for the N-terminal and C-terminal signal peptides. This PCR fragment was cloned into the SmaI restriction site of pUC18.

A fragment with NcoI/SstI was, in turn, cut out of this and cloned into an NcoI/SstI cut vector MA 01 (FIG. 1a), which contains the hordein-D promoter, followed by an SstI restriction site and by the Nos terminator. This gave rise to a construct containing a fragment consisting of hordein-D promoter, N-terminal thaumatin-signal peptide, thaumatin gene, C-terminal signal peptide and Nos terminator. A fragment with EcoRI and HindIII was, in turn, cut out of this construct. The EcoRi restriction site was filled by means of T4 polymerase and deoxy nucleotides before the cleavage with HindIII. This fragment was subsequently cloned into the vector MA 02 (FIG. 1b) which had been cut with NotI, followed by a filling reaction with T4 polymerase and deoxy nucleotides and, in another step, cut with HindIII. Subsequently, a fragment was cut out of this with EcoRI and HindIII and cloned into MA 03 (FIG. 1c). After the transformation of E. coli with the resulting vector and a plasmid preparation, this plasmid (RS 608, FIG. 1e) was transformed into AGL 1 cells which, in turn, were utilized for the transformation of immature barley embryos. The barley transformation was carried out according to Tingay et al. (supra).

### Example 3

Production and analysis of transgenic barley lines: in the case of each construct (607 with expression cassette (SEQ ID NO: 20), 608 with expression cassette (SEQ ID NO: 21), see Examples 1 and 2), 50 independent, transgenic barley lines were regenerated and cultivated in a greenhouse until they attained seed maturity. Individual kernel analyses of the mature seeds were carried out in order to select the lines with the highest yields of thaumatin.

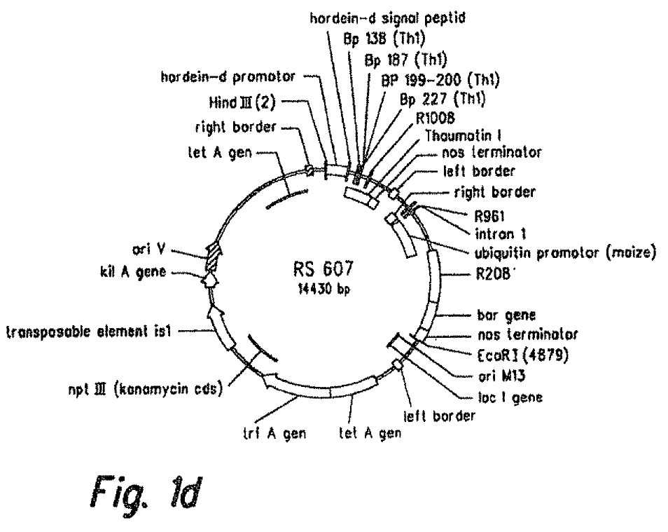

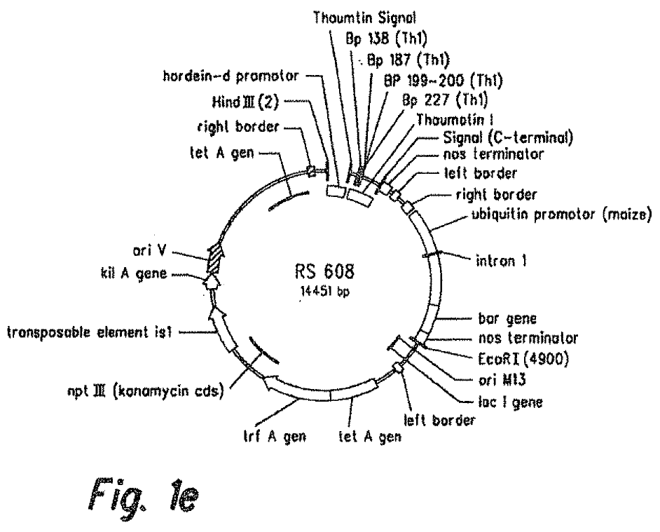

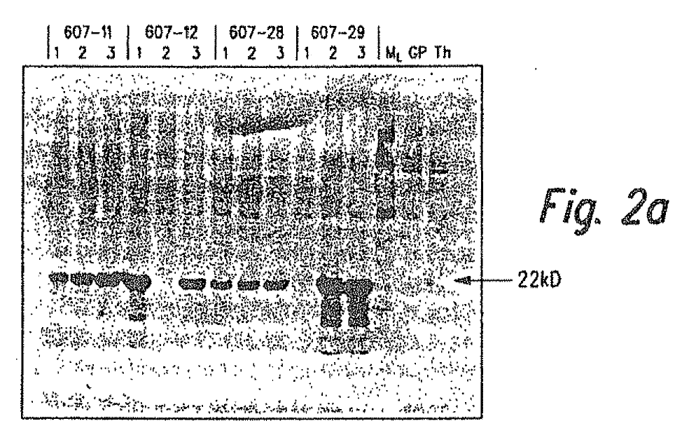

Mature kernels of the obtained transgenic barley lines were finely ground individually. The resulting flour was suspended in 80 mM citrate buffer (pH 3.0) and incubated for 30 minutes at 37° C. [98.6° F.]. The proteins thus extracted were obtained from the clear supernatant following centrifugation. The obtained extracts were neutralized, precipitated with acetone after the determination of the protein concentration, and then picked up in loading buffer and separated employing SDS-PAGE (sodium dodecyl sulfate polyacrylamide gel electrophoresis). Each time, 3 to 4 kernels per line were analyzed. The first examinations of the expression of thaumatin I in the barley lines were conducted by means of western blot analyses (FIG. 2a). Thaumatin-specific antibodies manufactured by the Abeam company were utilized for this purpose.

In accordance with the cleavage of the transgenes in the seeds of the TO lines, kernels with and without thaumatin expression were identified. Since the seeds of the transgenic barley lines accumulated high amounts of thaumatin, it was already possible to identify the heterologous protein in the SDS gels (FIG. 2b). Therefore, the highly expressing lines were selected employing SDS-PAGE in further examinations.

Highly expressing lines were further reproduced and homozygotic lines were identified. These were then employed for the further reproduction, for the development of purification methods, for the analysis of the recombinant proteins as well as for the production of malt extracts and instant powders.

### Example 4

### Purification of Thaumatin from Barley

The purification of pure thaumatin from the transgenic barley according to the invention can largely follow the protocol by Tate & Lyle (U.S. Pat. No. 4,011,206, U.S. Pat. No. 4,221,704). A fine flour of the kernel material of the transgenic barley according to the invention is mixed with water (acidified to a pH value of 2.7 to 3.0 through the addition of phosphoric acid, citric acid, formic acid, ascorbic acid and similar acids, which are used in food) at a 1:4 ratio.

The coarse components and starch are removed by means of filtration or sedimentation.

The extract already has a high concentration of thaumatin. If necessary, pure thaumatin can be obtained by means of ultrafiltration or ion-exchange chromatography.

In detail:

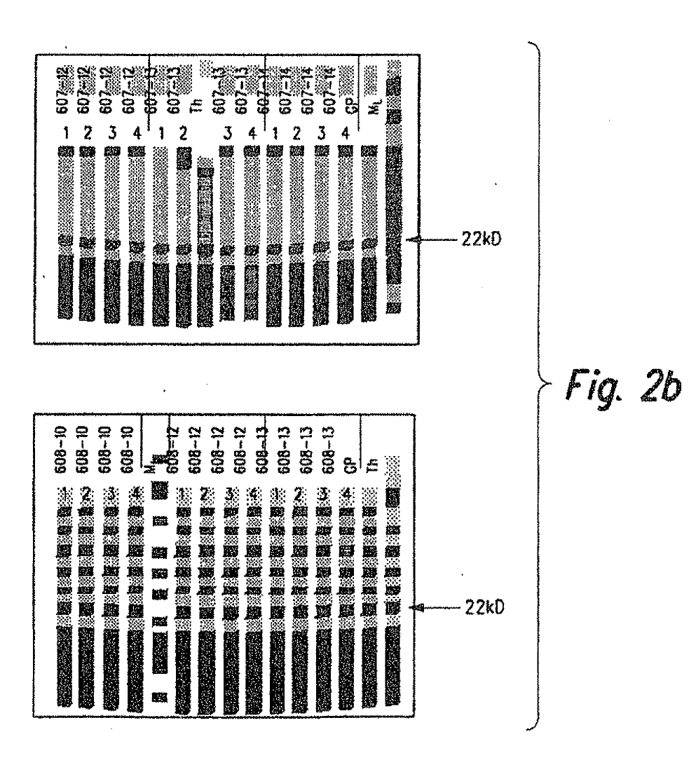

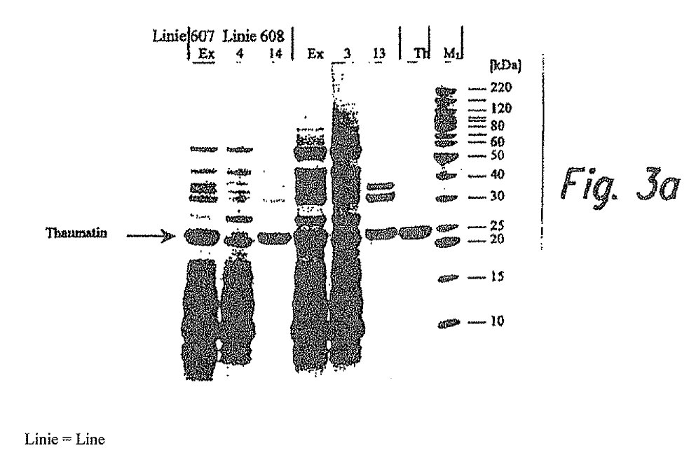

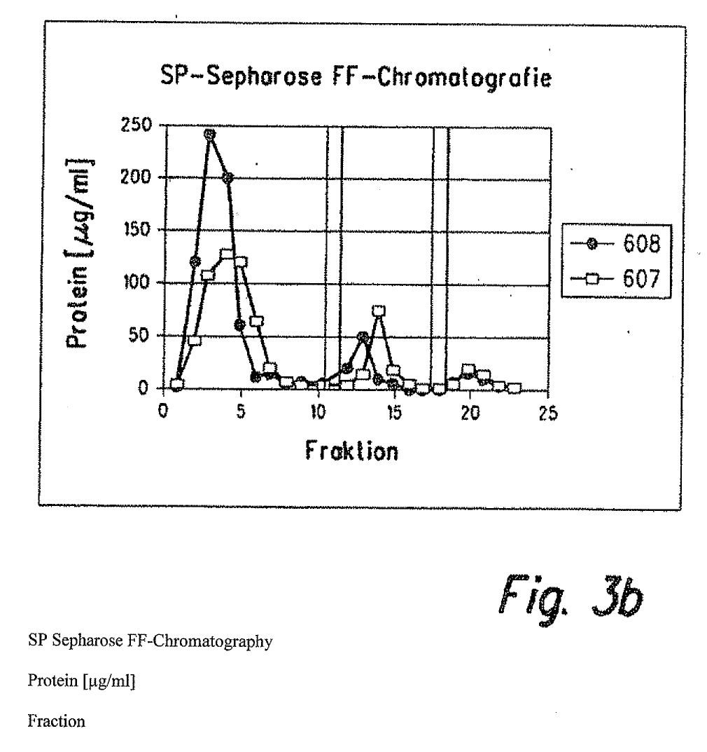

The following work was carried out according to standard methods, as described, for example, in “Methods in Enzymology 182” Methods in Enzymology Vol. 182: Guide to Protein Purification (M. P. Deutscher, ed.), Academic Press, San Diego, 1990. Mature kernels of the obtained transgenic barley lines (lines 607, 608, see FIG. 3a) were finely ground. The resulting flour was suspended in 80 mM citrate buffer (pH 3.0) and incubated for 30 minutes at 37° C. [98.6° F.]. The proteins thus extracted were obtained from the clear supernatant following centrifugation. The obtained extracts were demineralized by means of a Sephadex G25 M-gel filtration and rebuffered in 50 mM BICINE at a pH value of 8.8. The protein mixture containing thaumatin subsequently underwent cation-exchange chromatography. For this purpose, the extracts were applied onto an SP-sepharose fast-flow column (FIG. 2b) and pre-elution was carried out with 7 to 8 column volumes of starting buffer. The bound proteins were gradually eluted at a rising ion concentration (0.1 to 1 M NaCl) while maintaining the pH value under native conditions, and subsequently collected in fractions. The fractions containing thaumatin were identified by means of discontinuous SDS-PAGE analysis under reducing conditions.

### Example 5

### Preparation of Malt Powder

The method and the equipment employed here as well as the devices for the preparation of malt and mash are an integral part of brewing technology studies and are known to the person skilled in the art. In this context, reference is hereby made to “Die Technologie der Malzbereitung” [The technology of malt preparation] by Schuster/Weinfurtner/Narziss, 6^{th} edition, 1975, and to “Abriss der Bierbrauerei” [Compendium of beer brewing] by Narziss, 4^{th} edition, 1980. In order to provide a better understanding of the present invention, malt preparation according to the state of the art will be described in greater detail below.

In malt production, barley is processed to form malt in that the steps of soaking, germination, drying and curing are carried out in the malt house. During the soaking step, the kernels absorb up 48% water. For the subsequent germination, the germinating kernels are cooled with air kept at a temperature of about 15° C. [59° F.] under constant ventilation for 4 to 6 days.

The person skilled in the art speaks of so-called green malt after the germination. The malt kernels have a water content of about 44%.

The germination can be terminated through drying in a drying oven. The water is first removed at temperatures <60° C. [<140° F.] to lower the content to 12% and subsequently at about 85° C. [185° F.] to bring the water content down to 4.5%.

Cured malt having a water content of about 4.5% can be stored.

One kilogram of malt contains 1 gram to 3 grams, especially 2 grams or more, of active thaumatin, that is to say, the sweetening power of 1 kg of malt corresponds to that of about 3 kg to 9 kg of sucrose.

In order to be added to food, the cured malt is cleaned as the last step in the malt house. Either the malt is peeled or else a fine product having a low percentage of husks that is 60%-soluble is produced in a special grinding process.

The malt powder made of the transgenic barley according to the invention contains 1 kg to 3 kg, especially 2 kg, of thaumatin per ton of malt powder, in other words, one kilogram has the sweetening power of 3 kg to 9 kg of sucrose.

The malt powder is very well-suited as an additive in the manufacture of diet products. It can be used directly to make baking mixes, coffee and cocoa beverages, milk and chocolate products, candy, etc. The desired sweetness can be attained exclusively by the addition of the malt powder and no other sweeteners are necessary.

### Example 6

### Preparation of Malt Extract, Instant Powder

The preparation of a malt extract and instant powder is carried out according to the state of the art and is known to the person skilled in the art.

An extract is prepared by mixing malt and water at a weight ratio of, for instance, 1:5. In order to attain an optimal extraction of the thaumatin from the malt, the water is brought to a pH value between 2.7 and 3.0 through the addition of acids that are approved for food (citric acid, formic acid, phosphoric acid, etc.).

A concentrate from the malt extract (syrup, powder) and of an “instant powder” is prepared under vacuum or by means of freeze-drying according to standard methods that are known to the person skilled in the art.

Explanation of the Caption to FIGS. 2a, 2b, 3a:

FIG. 2a:

607-11, 607-12, 607-28, 607-29—several transgenic barley lines that express the thaumatin I gene under control of the hordein-D promoter with a hordein-D-signal
1, 2, 3—extracts from individual kernels of the appertaining lines
ML—kaleidoscope markers (Biorad)
GP—extract from kernels of a non-transgenic Golden Promise plant (control)
Th—extract from kernels of a non-transgenic barley line mixed with 5 ng of thaumatin I+II mixture (Sigma)

FIG. 2b:

SDS-PAGE of several independent transgenic TO lines that were transformed with the construct 607 or 608
607-12, 607-13, 607-14—several transgenic barley lines that express the thaumatin I gene under control of the hordein-D promoter with a hordein-D signal.
608-10, 608-12, 608-13—several transgenic barley lines that express the thaumatin I gene under control of the hordein-D promoter with the thaumatin-inherent N-terminal and C-terminal signals.
1, 2, 3, 4—extracts from individual grains of the appertaining 607 or 608 lines
ML—kaleidoscope markers (Biorad)
GP—extract from kernels of a non-transgenic Golden Promise plant (control)
Th—extract from kernels of a non-transgenic barley line mixed with 4.5 μg of thaumatin I+II (Sigma)

FIG. 3a:

Ex—flour extract (citrate buffer)
3, 4, 13, 14—aliquots of the corresponding eluate fractions (SP sepharose FF)
Th—thaumatin I+II (Sigma)
ML—protein standard

1. A transgenic plant from barley containing more than 2 grams of thaumatin in 1 kg of kernel material.

2. The transgenic plant from barley according to claim 1, containing active thaumatin.

3. The transgenic plant from barley according to claim 1, containing thaumatin I.

4. The transgenic plant from barley according to claims 1, containing thaumatin II.

5. The transgenic plant from barley according to claim 1, characterized in that the plant contains a protein selected from the group consisting of SEQ ID NO: 22-24 and SEQ ID NO: 28.

6. The transgenic plant from barley according to claim 1, having at least one polynucleotide that codes for thaumatin and that is stably integrated into the genome after its transformation.

7. The transgenic plant from barley according to claim 6, characterized in that the polynucleotide is selected from the group consisting of SEQ ID NO: 25-27 and SEQ ID NO: 29.

8. The transgenic plant from barley according to claim 6, containing promoters and optionally other secondary sequences that serve for the controlled expression of thaumatin.

9. The transgenic plant from barley according to claim 6, containing endosperm-specific promoters.

10. The transgenic plant from barley according to claim 6, containing endosperm-specific promoters selected from the group consisting of hordein-B, -C, -D, -G promoters; SEQ ID NO: 1; gamma hordothionine promoters; the Gt1 promoter; and the Glu-lD-1 promoter.

11. The transgenic plant from barley according to claim 6, containing signal sequences for targeting into the endoplasmic reticulum of the endosperm cells.

12. The transgenic plant from barley according to claim 6, containing signal sequences selected from the group consisting of hordein-B, hordein-C, hordein-D, hordein-G, gamma hordothionines having SEQ ID NO: 2-8, SEQ ID NO: 16 and SEQ ID NO: 17.

13. The transgenic plant from barley according to claim 11, containing signal sequences from the group consisting of SEQ ID NO: 9-15.

14. The transgenic plant from barley according to claim 8, characterized in that the controlled expression takes place in the presence of protein disulfide isomerases.

15. A vector containing a construct or an expression cassette comprising at least one polynucleotide that codes for thaumatin, at least one endosperm-specific promoter, and optionally signal sequences for targeting into the endoplasmic reticulum of endosperm cells.

16. The vector according to claim 15, containing a construct or expression cassette according to SEQ ID NO: 21-22.

17. A transformed plant cell or transformed plant tissue or transformed protoplast having a polynucleotide that is stably integrated into the genome after its transformation, according to claim 6.

18. (canceled)

19. (canceled)

20. Kernel material or seeds of a transgenic plant from barley according to claim 1.

21-27. (canceled)

28. A polynucleotide that codes for the protein of SEQ ID NO: 28.

29. The polynucleotide of claim 28 wherein said polynucleotide is SEQ ID NO: 29.


SEQUENCE LISTING (33 sequences)

SEQ ID NO: 1 - DNA, 434 bp, Hordeum vulgare
cttcgagtgcccgccgatttgccagcaatggctaacagacacatattctgccaaaaccccagaacaataatcacttctcgtagatgaagagaacagaccaagatacaaacgtccacgcttcagcaaacagtaccccagaactaggattaagccgattacgcggctttagcagaccgtccaaaaaaactgttttgcaaagctccaattcctccttgcttatccaatttcttttgtgttggcaaactgcacttgtccaaccgattttgttcttcccgtgtttcttcttaggctaactaacacagccgtgcacatagccatggtccggaatcttcacctcgtccctataaaagcccagccaatctccacaatctcatcatcaccgagaacaccgagaaccacaaaactagagatcaattcattgacagtccaccgag

SEQ ID NO: 2 - DNA, 57 bp, Hordeum vulgare
atgaagaccttcctcatctttgcactcctcgccattgcggcaacaagtacgattgcg

SEQ ID NO: 3 - DNA, 57 bp, Hordeum vulgare
atgaagaccttcctcatctttgcactcctcgtcattgcggcaacaagtacgattgca

SEQ ID NO: 4 - DNA, 84 bp, Hordeum vulgare
atggctaagcggctggtcctctttgtggcggtaatcgtcgccctcgtggctctcaccaccgctgaacgtgagatcaatgggaac

SEQ ID NO: 5 - DNA, 63 bp, Hordeum vulgare
atggctaagcggctggtcctctttgtggcggtaatcgtcgccctcgtggctctcaccaccgct

SEQ ID NO: 6 - DNA, 57 bp, Hordeum vulgare
atgaagatcttaatcatccttacaatccttgccatggcgactaccttcgccacctct

SEQ ID NO: 7 - DNA, 60 bp, Hordeum vulgare
atgaagacgttcctcacctttgtcctccttgccatggcgatgagcatcgtcactaccgct

SEQ ID NO: 8 - DNA, 102 bp, Hordeum vulgare
atggagttcaagacgaaggcgaccgtgtgcgcggcgctgctggtgctgctcctgctttcctcctacgacggcggcgacggcggcgtcggcgtggcggaggcg

SEQ ID NO: 9 - protein, 19 aa, Hordeum vulgare
MKTFLIFALLAIAATSTIA

SEQ ID NO: 10 - protein, 19 aa, Hordeum vulgare
MKTFLIFALLVIAATSTIA

SEQ ID NO: 11 - protein, 28 aa, Hordeum vulgare
MAKRLVLFVAVIVALVALTTAEREINGN

SEQ ID NO: 12 - protein, 21 aa, Hordeum vulgare
MAKRLVLFVAVIVALVALTTA

SEQ ID NO: 13 - protein, 19 aa, Hordeum vulgare
MKILIILTILAMATTFATS

SEQ ID NO: 14 - protein, 20 aa, Hordeum vulgare
MKTFLTFVLLAMAMSIVTTA

SEQ ID NO: 15 - protein, 34 aa, Hordeum vulgare
MEFKTKATVCAALLVLLLLSSYDGGDGGVGVAEA

SEQ ID NO: 16 - DNA, 66 bp, Thaumatococcus daniellii
atggccgccaccacttgcttcttcttcctcttccccttcctcctcctcctcacgctctcccgcgct

SEQ ID NO: 17 - DNA, 18 bp, Thaumatococcus daniellii
cttgaacttgaagacgag

SEQ ID NO: 18 - protein, 22 aa, Thaumatococcus daniellii
MAATTCFFFLFPFLLLLTLSRA

SEQ ID NO: 19 - protein, 6 aa, Thaumatococcus daniellii
LELEDE

SEQ ID NO: 20 - DNA, 1434 bp, Artificial
  Synthetic expression cassette
aagcttgcatgcggctgcaggtcgactctagaggatcccttcgagtgcccgccgatttgccagcaatggctaacagacacatattctgccaaaaccccagaacaataatcacttctcgtagatgaagagaacagaccaagatacaaacgtccacgcttcagcaaacagtaccccagaactaggattaagccgattacgcggctttagcagaccgtccaaaaaaactgttttgcaaagctccaattcctccttgcttatccaatttcttttgtgttggcaaactgcacttgtccaaccgattttgttcttcccgtgtttcttcttaggctaactaacacagccgtgcacatagccatggtccggaatcttcacctcgtccctataaaagcccagccaatctccacaatctcatcatcaccgagaacaccgagaaccacaaaactagagatcaattcattgacagtccaccgagatggctaagcggctggtcctctttgtggcggtaatcgtcgccctcgtggctctcaccaccgctgccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaacggtggcaaaatctgggcccgcaccgactgctatttcgacgacagcggcagcggcatctgcaagaccggcgactgcggcggcctcctccggtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcctgagagctcgctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgg

SEQ ID NO: 21 - DNA, 1455 bp, Artificial
  Synthetic expression cassette
aagcttgcatgcggctgcaggtcgactctagaggatcccttcgagtgcccgccgatttgccagcaatggctaacagacacatattctgccaaaaccccagaacaataatcacttctcgtagatgaagagaacagaccaagatacaaacgtccacgcttcagcaaacagtaccccagaactaggattaagccgattacgcggctttagcagaccgtccaaaaaaactgttttgcaaagctccaattcctccttgcttatccaatttcttttgtgttggcaaactgcacttgtccaaccgattttgttcttcccgtgtttcttcttaggctaactaacacagccgtgcacatagccatggtccggaatcttcacctcgtccctataaaagcccagccaatctccacaatctcatcatcaccgagaacaccgagaaccacaaaactagagatcaattcattgacagtccaccgagatggccgccaccacttgcttcttcttcctcttccccttcctcctcctcctcacgctctcccgcgctgccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaacggtggcaaaatctgggcccgcaccgactgctatttcgacgacagcggcagcggcatctgcaagaccggcgactgcggcggcctcctccggtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcccttgaacttgaagacgagtaagagctcgctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgg

SEQ ID NO: 22 - protein, 207 aa, Thaumatococcus daniellii
ATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 23 - protein, 207 aa, Thaumatococcus daniellii
ATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 24 - protein, 235 aa, Thaumatococcus daniellii
MAATTCFFFLFPFLLLLTLSRAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTALELEDE

SEQ ID NO: 25 - DNA, 620 bp, Thaumatococcus daniellii
gccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaacggtggcaaaatctgggcccgcaccgactgctatttcgacgacagcggcagcggcatctgcaagaccggcgactgcggcggcctcctccggtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgc

SEQ ID NO: 26 - DNA, 621 bp, Thaumatococcus daniellii
gccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaagggtggcaaaatctgggcccgcaccgactgctatttcgacgacagcggccgcggcatctgccggaccggcgactgcggcggcctcctccagtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcc

SEQ ID NO: 27 - DNA, 635 bp, Thaumatococcus daniellii
ccttcgagatcgtcaaccgctgctcctacaccgtgtgggcggccgcctccaaaggcgacgccgccctggacgccggcggccgccagctcaactcgggagagtcctggaccatcaacgtagaacccggcaccaagggtggcaaaatctgggcccgcaccgactgctatttcgacgacagcggccgcggcatctgccggaccggcgactgcggcggcctcctccagtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcccttgaacttgaagacgag

SEQ ID NO: 28 - protein, 235 aa, Artificial
  Synthetic protein sequence
MAATTCFFFLFPFLLLLTLSRAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTALELEDE

SEQ ID NO: 29 - DNA, 705 bp, Artificial
  Synthetic nucleotide sequence
atggccgccaccacttgcttcttcttcctcttccccttcctcctcctcctcacgctctcccgcgctgccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaacggtggcaaaatctgggcccgcaccgactgctatttcgacgacagcggcagcggcatctgcaagaccggcgactgcggcggcctcctccggtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcccttgaacttgaagacgag

SEQ ID NO: 30 - DNA, 24 bp, Artificial
  Synthetic primer
gccaccttcgagatcgtcaaccgc

SEQ ID NO: 31 - DNA, 28 bp, Artificial
  Synthetic primer
ggcagtagggcagaaagtgaccctgtag

SEQ ID NO: 32 - DNA, 24 bp, Artificial
  Synthetic primer
atggccgccaccacttgcttcttc

SEQ ID NO: 33 - DNA, 33 bp, Artificial
  Synthetic primer
agagctcttactcgtcttcaagttcaagggcag